(19) United States
(12) Patent Application Publication
(10) Pub. No.: US 2021/0015720 A1
(43) Pub. Date: Jan. 21, 2021

(54) SKIN AND HAND CLEANSING COMPOSITIONS COMPRISING SCRUBBING MATERIALS

(71) Applicant: DEB IP LIMITED, DENBY, DERBYSHIRE (GB)

(72) Inventors: NICOLE KAMPS, KREFELD (DE); MARCEL VEEGER, KREFELD (DE)

(21) Appl. No.: 16/603,731

(22) PCT Filed: May 18, 2018
(86) PCT No.: PCT/GB2018/051346

§ 371 Date: Oct. 8, 2019

(30) Foreign Application Priority Data
May 19, 2017 (GB) 1708104.3

(51) Int. Cl. A61K 8/04; A61K 8/73; A61K 8/92; A61K 8/42; A61K 8/46; A61Q 19/10

(52) U.S. Cl. CPC A61K 8/046; A61K 8/73; A61K 8/922; A61K 8/42; A61K 8/463; A61K 2800/87; A61K 2800/524; A61K 2800/48; A61K 2800/596; A61K 2800/28; A61Q 19/10

(57) ABSTRACT

The application describes foamable skin and hand cleansing compositions, comprising the following : a) 0.5 to 10% by weight of sulphated castor oil; b) 0.1 to 1% by weight of xanthan gum; c) 0 to 5% by weight of at least one non-aqueous solvent, typically an organic solvent; d) 1 to 30% by weight of at least on surfactant; e) 0 to 3% by weight of at least one emulsifier; f) 1 to 20% by weight of at least one scrubbing agent; g) 0 to 2% by weight of at least one preservative; h) 0 to 2% by weight of at least one pH modifier; i) 0 to 10% by weight of auxiliaries and/or additive; and j) water to make up 100% by weight.

The invention relates to foamable skin and hand cleansing compositions comprising scrubbing materials, and to their use.

Liquid skin and hand cleansing formulations are generally known in the art. They are conventionally provided in containers that are poured or have pumps to pump the liquid compositions onto the skin to be cleaned. Such liquid soaps often have very good cleaning efficiency.

Formulations which are foamed before being placed on the skin or hands are also generally known in the art. Foams tend to be much easier to spread than the corresponding liquid and in addition there is much less waste due to splashing or run-off since the foam has a much higher surface tension than a liquid. One problem associated with foams compared with liquid soaps is they tend to have a lower cleaning efficiency. However, foams have a much higher surface area than un-foamed liquid, so if the cleaning efficiency of the foam can be improved, it is possible to produce foams with the same cleaning power, as obtained with un-foamed liquid but which require much less of the initial liquid to be used.

With the addition of mechanical scrubbers, also known as scrubbing agents, the cleaning performance of liquid soaps can be enhanced. Liquid soaps with suspended scrubbing agents tend to have a viscosity range at their lower end that is higher than that of basic liquid soaps. In order to provide the structure to suspend the mechanical scrubbers of an appropriate size to provide cleaning benefits, these formulations have tended to have a critical strain force (or yield value) superior or equal to 30 dynes/cm², for example associated with non-Newtonian rheological behaviour. Critical strain force is defined as the stress which must be applied before flow will start, although related to viscosity, it is more dependent on the characteristics of the rheological additive used.

As indicated above, the use of foaming hand cleansers offer many benefits over liquid hand soaps. Typically, such foamable soaps are not capable of suspending mechanical scrubbers of sufficient size to provide cleaning benefits.

WO 2014/019944 describes a foamable gritty foam composition comprising particulate mechanical scrubbers with particles sized from 100 microns to about 800 microns as capable of being foamed through a non-aerosol, or unpressurised foam dispenser. The formulation describes using between 0.5% weight for weight to about 30% weight for weight of a solvent, such as D-limonene or sunflower methyl ester. A wide range of thickeners are described, including those as diverse as xanthan gum, guar gum, quaternised guar gum, alginate, bentonite and fumed silica. One problem associated with such prior art formulations is that they comprise non-aqueous solvents, such as D-limonene, sunflower oil methyl esters or other organic solvents. Such organic solvents are not always acceptable to consumers. Currently there is a need to try and reduce or remove such solvents.

Many prior art formulations use carbomers. Such formulations typically have a pH of 5.5-8. The ability to produce a formulation with a pH lower than 5.5, would allow the use of alternative preservatives and produce formulations with improved cleansing properties.

WO 2005/107699 describes an alternative foaming formulation with particles suspended within it that utilises carbomers and a solution of sodium chloride as a preferred system. Such thickeners require a predetermined pH or range of pH to thicken the liquid if pH of the liquid falls outside that range the thickener no longer acts as a thickener.

Other formulations using carboxymethylcellulose (CMC) are known in the art (US 2012/02027304). CMC provides a high viscosity with reduced foaming and instability problems.

The Applicant has identified that it is possible to use xanthan gum, and especially smooth flow xanthan gums, in combination with sulphated castor oils, to produce skin and hand cleansing compositions comprising scrubbing agents.

The invention provides a foamable skin and hand cleansing composition comprising the following components based on the total composition of the cleansing composition:

a) 0.5 to 10% by weight of sulphated castor oil;

b) 0.1 to 1% by weight of xanthan gum;

c) 0 to 5% by weight of at least one non-aqueous solvent, typically an organic solvent;

d) 1 to 30% by weight of at least one surfactant;

e) 0 to 3% by weight of at least one emulsifier;

f) 1 to 20% by weight of at least one scrubbing agent;

g) 0 to 2% by weight of at least one preservative;

h) 0 to 2.5% by weight of at least one pH modifier;

i) 0 to 10% by weight of auxiliaries and/or additive; and

j) water to make up 100% by weight.

Xanthan gums are polysaccharides produced by microorganisms of the genus Xanthomonas, and in particular Xanthomonas campestris.

The gum is typically obtained by fermentation of the microorganism and is generally used for a wide range of different uses in the food and chemical industries. Thickening compositions using xanthan gum tend to have a non-uniform or “chunky” flow characteristic, typical to that found with tomato ketchup. Whilst such conventional xanthan gum may be used, the Applicant has unexpectedly found that so-called “smooth-flow” xanthan gums produce better foamable compositions because they have lower viscosity than standard xanthan gums. Such smooth-flow xanthan gums are described in, for example, EP 0005030 and U.S. Pat. No. 4,263,399. Such smooth-flow xanthan gums are available from CP Kelco, under the name of, for example, Keltrol®CG-SFT. Such smooth-flow xanthan gums are typically formed and made using high shear conditions that produces a material that is generally homogeneous, compared with native uneven xanthan gums. The material also has lower extensional viscosity than conventional xanthan gum and is stable under high shear conditions compared to conventional xanthan gum which shows reduced viscosity on being subjected to shearing.

Typically, additional thickeners such as cellulose gums are not used. The addition of cellulose gums, for example, was found to reduce foamability.

Typically, the composition has a viscosity of 500 to 5000 mPa·s. More typically this is less than 3500 mPa·s, between 800-2000 mPa·s, especially 800-1300 mPa·s. Above 3500 mPa·s or above 2000 mPa·s produces reduced foaming.

Typically the composition has a yield value of at least 30 dynes/cm² up to typically 200 dynes/cm², more typically 50 to 100 dynes/cm² or 80 dynes/cm².

The pH of the composition may be below 6, especially 5.5 or below, more typically above pH 3, or above pH 4.

Sulphated (also known as sulphonated) castor oil is castor oil that has been treated with, for example, sulphuric acid to produce an anionic surfactant.

Typically, the composition comprises 1 to 8% by weight, 1 to 5.5, more typically 3 to 5% or 3 to 3.5% by weight of sulphated castor oil. Sulphated castor oil acts as a surfactant and improves the stability of the formula. The sulphated castor oil may be provided as substantially pure material or containing, for example, 70% castor oil pure sulphated castor oil.

Typically 0.1 to 1, 0.2 to 0.9 more typically 0.4 to 0.8% by weight of xanthan gum may be used.

“Conventional” xanthan gums are those which have not been made using higher shear conditions, for example as described above. Application of high shear rates to such gums reduces the viscosity of such gums.

Both conventional xanthan gums and smooth flow xanthan gums, or mixtures thereof may be used.

However, where conventional xanthan gums, the Applicant identified that higher levels of xanthan gum produce higher viscosities which inhibit foam formation.

Accordingly, the amount of xanthan gum, for example conventional xanthan gum (but alternatively smooth xanthan gum or mixtures thereof) may be limited to 0.1-0.5%, more typically 0.35-0.45% by weight. Typically, this reduces the viscosity to below 4000 mPa·s.

Typically less than 5%, less than 3% or less than 2% by weight of at least one non-aqueous solvent such an organic solvent is used, more typically no non-aqueous solvent is used. Non-aqueous solvent may be one or both of D-limonine and sunflower oil methyl ester. Other solvents include glycol ethers, esters, alcohols, other terpenes and aromatic-free white spirit. Non aqueous solvents such as D-limonine may be omitted from the formulation.

The composition typically comprises 2 to 30% by weight, more typically 2 to 20%, or 3 to 10% by weight of the additional surfactant (component d)).

Surfactants include for example:

i) Amphoteric Surfactants

The amphoteric surfactant may be any one or combination of betaines, acyl ethylene diamines, amino-acids derivatives, imidazolines. Alternatively, the amphoteric surfactant may be any one or combination of acylamphoacetate, acylamphodiacetate, acylamphodipropionate, sodium cocoglycinate, sodium alkyliminodipropionate, cocamidopropyl betaine, sodium cocoamphoacetate.

The betaine may be any one or combination of coco betaine and cocamidopropyl betaine.

ii) Non-Ionic Surfactants

The non-ionic surfactant may be any one or combination of glucosides, ethoxylated fatty alcohols, ethoxylated fatty acids, saccharose esters, sorbitan esters, alkanolamides, glycerol alkyl esters, polyoxyethylene glycol alkylphenol esters.

iii) Anionic Surfactants

The anionic surfactant may be any one or combination of lauryl sulphates, lauryl ether sulphates, sulphosuccinates, carboxylates (i.e. sodium oleate), carboxylic acid esters (i.e. sodium dilaureth citrate), alkyl sulfate (i.e. sodium lauryl ether sulfate, ammonium alkyl sulfate, alkyl and alkyl-aryl sulfonates (i.e. sodium dodecyl benzene sulfonate), sulfosuccinates (i.e. disodium lauryl ether sulfosuccinate), isethionates (i.e. sodium cocoyl isethionate, ammonium cocoyl isethionate), taurates (i.e. sodium methyl cocoyl taurate, sodium methyl oleoyl taurate)acyl glutamates (i.e. sodium lauroyl glutamate, sodium cocoyl glutamate, disodium cocoyl glutamate), sarcosinate (i.e. cocoyl sarcosinate), alkylpolyglucosides (i.e. decyl glucoside, sodium lauryl glucose carboxylate, caprylyl/capryl glucoside).

The surfactants more typically include one or more fatty acid glucamides.

The fatty acid glucamide typically as the general formula:

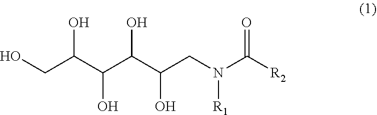

where:

R¹=H or a C1 or C4 alkyl, most typically H or —CH₃, especially —CH₃

R²=C₆ to C₂₀ linear or branched hydrocarbon residue, most typically C₈ to C₁₀ linear or unbranched hydrocarbon residue. The hydrocarbon residue may be saturated or unsaturated.

Mixtures of the glucoamines may be used. For example, the glucamide may be a mixture of capryloyl/caproyl methyl glucamide; lauroyl/myristoyl methyl glucamide; or cocoyl methyl glucamide. Mixtures of these may also be used, for example, a mixture of cocoyl methyl glucamide and capryloyl/caproyl methyl glucamide may be used. Cocoyl methyl glucamide comprises fatty acid residues from coconut oil.

The surfactant may be sodium laureth sulfate or disodium laureth sulfosuccinate. Mixtures of one or more fatty acid glucamides, sodium laureth sulfate (SLS) and disodium laureth sulfosuccinate (DLS) may be used, or indeed mixtures of other surfactants.

Capryloyl/caproyl methyl glucamide optionally mixed with sodium laureth sulfate and/or disodium laureth sulfosuccinate may be used.

The amount of fatty acid glucamide may be between 2.0 and 5.5% by weight, especially 2.5 to 3.5% by weight. SLS may be present in 3.0 to 8.0% by weight. DLS may be present between 2.0 and 4.5% by weight.

Typically the composition may comprise 0.5 to 2.5% by weight, more typically 1.0 to 2.0% of emulsifier. The emulsifier may be selected from those generally known in the art including PEG-18, castor dioleate, polyglycerin 4-caproate, d-n-octylether lauryl glucoside, caprylyl glycol, sorbitan sequicaprate, hydrogenated castor oil, PEG-10 glyceryl oleate and gemini surfactants and mixtures thereof. Gemini surfactants are a family of surfactant molecules possessing more than one hydrophobic tail and hydrophilic head group. Gemini surfactants usually have better surface-active properties than corresponding conventional surfactants of equal chain length. For example, they have the hydrophobic to hydrophilic head groups connected to one another via a spacer. These are described in, for example, a review article by S. K. Hate and S. P. Moulik, Current Science, Vol 82(9) (2002) 1101-1111. Typically the emulsifier is glyceryl oleate citrate, (commercially available as Dermofeel®Easymuls Plus, Dr Straetmans GmbH), or polyglyceryl 3 caprate (commercially available under the name Tegosoft®PC 31, Evonik Industries AG).

The composition may comprise 5 to 15% by weight of scrubbing agent. The scrubbing agent is particulate and is any one or combination of a vegetable based scrubbing agent, a synthetic based scrubbing agent and a mineral based scrubbing agent, and the particles may have a size in a range from about 100 microns to about 800 microns, or in a range from about, or in a range from about 200 to about 700, or in a range from about 300 to about 500 microns. The particles may have any size in this range, or be in any narrow range than 100 to 800 microns, or there may be a mixture of any size of particles in this broad range present in the composition. The vegetable based scrubbing agent may be any one or combination of cornmeal, olive stone, walnut shells, ground fruit stones, ground corn meal, ground fruit shells. The synthetic based scrubbing agent may be any one or combination of polyethylene and polypropylene. The mineral based scrubbing agent may be any one or combination of ground shellfish, pumice and silica.

The composition may comprise 0.002 to 2% by weight of at least one preservative. The preservative may be an organic acid, such as sodium benzoate or potassium sorbate.

The preservative may be present in an amount 0.1 to 2.0% or 0.1 to 1.5%, or 0.1-0.9% or 0.5-0.9% by weight.

pH modifiers may be used to adjust the pH. These include organic acids such as citric acid and lactic acid.

Typically, less than 0.5%, less than 0.4%, less than 0.2%, less than 0.1% wt or no additional thickeners are included. Such additional thickeners include polysaccharides such as carboxymethylcellulose (CMC). Typically, no additional thickener, such as CMC, in incorporated.

The formulation may comprise 0 to 10% by weight of auxiliaries and/or other additives, these include, for example, colourants, salts (such as sodium chloride or monosodium phosphate), oleic acid, pearlessing agents and, fragrances. Typically these are present in an amount of 1 to 9%, 3-7% or 5% by weight.

Water is added to make the total percentage weight 100%.

The invention also provides hand pumping apparatus comprising compositions of the invention. Such hand pumping apparatus include those described for example, in WO 2005/107699. Typically the apparatus uses a hand pumping dispenser, rather than a pre-pressurised dispenser.

Methods of washing skin using the compositions of the invention are also provided. This typically includes expelling the composition through a foaming apparatus to produce a foam, and applying the foam to a portion of the skin of the subject. This may then be washed off using water, together with at least a portion of any dirt found on the skin. Skin is typically the hands and/or the face of a subject.

The invention will now be described by way of example with reference to the following table. The table shows examples of different formulae and effect of different additives on cleansing foamability and stability of the foam.

Cleansing performance was typically carried out using the following method:

Foam Production

The formulations described below were tested by evaluating the Foam quality as a ratio between the g product/ml foam. This was aimed to be typically 1:4 or even better 1:5 but not lower or equal to 1:2.

Cleansing Performance

Cleansing performance was typically carried out using the following method:

The test model of the hand washing test with standardised soiling or paint gives information about the cleaning effect of the products to be tested. For relevance in practice, it is necessary that all subjects have a characteristic skin structure on the palms of the hands caused by manual work. Using one product at time, the following test is carried out in the morning and afternoon:

Test Procedure With Water:

0.5 g of soiling (model soiling, practice soiling or paint) is distributed on the palm and the back of the hand and rubbed in.

Rub in and leave to dry within 2 min.

1.2 g of cleansing composition are applied and rubbed in

1 ml of water is added twice within 1 min.

Rinse under hand warm running tap water and remove as much or all of the soil as possible within 30 s

Visual assessment of the residual soiling (RS) on the back of the hand and the palm according to the scale, see below.

Test Procedure Without Water:

0.5 g of soiling (model soiling, practice soiling or paint) are distributed on the palm and on the back of the hand and rubbed in

Leave to dry for 1½ min

1.2 g of cleaning composition are applied and rubbed in

Using a cellulose paper, the soiling on the hand surfaces is removed together with the product

Visual assessment of the residual soiling (RS) on the back of the hand and the palm according to the scale, see below.

0=clean 5=no cleaning effect (graduation in steps of 0.5 possible).

The percentage cleaning effect is calculated according to the following formula:

$$cleaning effect [ \% ] = \frac{10 - ( {\overline{RS}}_{palm} + {\overline{RS}}_{back} )}{10} * 100 \%$$

RSₚₐₗₘ=mean value of the residual soiling on palms of n measurement series (subjects).

RS_{back}=mean value of the residual soiling on backs of hands of n measurement series (subjects).

Since the deterioration of the cleaning effect has a broader variation range, an absolute deviation of 5% between two measurement series is allowable.

Composition of a suitable model soiling:

Flame soot: 5.42%

Iron oxide (Fe₂O₃): 0.72%

In the Examples below the amount of sulphonated castor oil used is the amount of 100% pure sulphonated castor oil. Examples are wt %.

Keltrol is “smooth” xanthan gum. Jungbunzlauer “conventional” xanthan gum.

Typically water was added to a vessel and mixed with the xanthan gum by stirring. Scrubbing agents and preservatives were then added followed by the surfactants; including the sulphonated castor oil, then the emollients and perfume. The pH was adjusted using the pH modifiers.

A list of components used in the examples by trade name and INCI name follows, along with examples of the invention and comparative examples.

The following examples provide particularly good hand cleansing, foamability and stability: B7 to B16, C1 to D6, D8 to D10, E2 to F3, O1 and O1.1 to O1.3.

| Trade Name | INCI Name |
|---|---|
| Water | Aqua |
| Yield improvement | |
| Keltrol CG-SFT | Xanthan Gum |
| Sucraclear HC-31 | Cellulose Gum, Chondrus Crispus |
| | (Carrageenan) Extract, Ceratonia Siliqua |
| | Gum, Sucrose |
| Jungbunzlauer FEDCS-PC | Xanthan Gum |
| Simulgel FL | Hydroxyethyl Acrylate/Sodium |
| | Acryloyldimethyl Taurate Copolymer; |
| | Isohexadecane; Polysorbate 60 |
| Exilva | |
| Aristoflex TAC | Ammonium Acryloyldimethyltaurate/ |
| | Carboxyethyl Acrylate Crosspolymer |
| Actigum VSX 20 | sclerotium gum and xanthan gum |
| Kelcogel CG-LA | Gellan gum |
| Rhodicare S | Xanthan Gum |
| Walocel CRT 50.000 PA 07 | Cellulose Gum |
| Nonionics | |
| Gluco Tain Clear | Capryloyl/Caproyl Methyl Glucamide |
| Gluco Tain Care | Cocoyl Methyl Glucamide |
| Rewopal LA 6 | Laureth 6 |
| Marlipal 24/70 | Trideceth 7 |
| Mackamine LA | Lauramine Oxide |
| Bio Saponins | Saponins |
| Steposol MET 10 U | N,N-dimethyl 9-decanamide |
| Betaine Coco Base | Cocoamidopropyl Betaine |
| Anionics | |
| SLES | Sodium Laureth Sulfate |
| Sulfated Castor Oil 75 WS | Sulfated Castor Oil |
| Rewopol SBF A 30 B | Disodium Laureth Sulfosuccinate |
| Protelan LS9011 | Natrium-N-lauroylsarkosinat |
| Greenbentin MLS/100 | Fatty acids C12-16 and C18-unsaturated, |
| | esters with polyethylene glycol mono-Me |
| | ether |
| Eucarol Age SS | Disodium alkylpolyglucose |
| TEA Laurylsulfat | TEA-Lauryl Sulphate |
| Emulsifieres/Stabiliser | |
| Dermofeel Easymuls Plus | Glyceryl Oleate Citrate |
| Dermosoft Octiol | Caprylyl Glycol |
| Versatil MPC | Methylpropanediol; Phenoxyethanol; |
| | Caprylyl Glycol |
| Antilsoft SC | Sorbitan Sesquicaprylate |
| Oxypon 401 | PEG-9 Cocoglycerides |
| Glycerin pflanzlich 99.5% | Glycerin |
| Polyglycerin-6 | Glycerin |
| Cetiol OE | di-n-octyl Ether |
| Glucopon 650 EC/HH | Lauryl Glucoside |
| Cosmacol ELI | C12-13 Alkyl Lactate |
| Tegosoft PC 31 | Polyglyceryl 3 Carprate |
| Preservative | |
| Organic acid | Sodium Benzoate |
| Organic acid | Potassium Sorbate |
| Scrubber | |
| Cornmeal | Corn (Zea Mays) Meal |
| Olive Stone Granules | Olea Europaea (olive) |
| | Seed Powder |
| Auxiliaries | |
| Fragrance | Parfum (Fragrance) |
| Palmera A1813 | Oleic Acid |
| pH Adjuster | Citric acid |
| Mononatriumphosphat calc. | Mononatriumphosphat calc. |
| Sodiumchlorid solution 15% | Sodium Chloride |
| Performance | |

Cleansing effect (Method as described) as requested > 70%
Foamability - Foamquality (Methode as described) as requested > 3.9
Stability (Method as described) as requested min+

| Trade Name | A1 | B1 | B2 | B3 | B4 | B5 | B6 | B7 | B8 | B9 | B10 |
|---|---|---|---|---|---|---|---|---|---|---|---|
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | 0.10 | 0.30 | 0.30 | 0.30 | 0.30 | 0.30 | 0.30 | 0.30 | 0.30 | 0.30 | 0.30 |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | | | | | | | | | | | |
| Actigum VSX 20 | | | | | | | | | | | |
| Kelcogel CG-LA | | | | | | | | | | | |
| Rhodicare S | | | | | | | | | | | |
| Walocel CRT 50.000 PA 07 | | | | | | | | | | | |
| Nonionics | | | | | | | | | | | |
| Gluco Tain Clear | | | | | | | 2.20 | 3.30 | 3.30 | 3.30 | 3.30 |
| Gluco Tain Care | | | | | | | | | | | |
| Rewopal LA 6 | 6.00 | | | | | | 4.00 | | | | |
| Marlipal 24/70 | | | | | | | | | | | |
| Mackamine LA | | | | | | | | | | | |
| Bio Saponins | | | | | | | | | | | |
| Steposol MET 10 U | | | | | | | | | | | |
| Betaine Coco Base | | | | | | | | | | | |
| Anionics | | | | | | | | | | | |
| SLES | 3.64 | 3.38 | 3.64 | 6.50 | 10.40 | 13.00 | 6.50 | 3.37 | 3.38 | 5.20 | 5.20 |
| Sulfated Castor Oil 75 WS | 3.00 | 3.00 | 3.00 | 3.00 | 3.00 | 3.00 | 3.00 | 3.00 | 3.00 | 2.00 | 3.00 |
| Rewopol SBF A 30 B | 2.10 | 1.20 | 2.10 | 12.60 | 3.50 | 3.50 | | | 1.75 | | |
| Protelan LS9011 | | | | | | | | | | | |
| Greenbentin MLS/100 | | | | | | | | | | | |
| Eucarol Age SS | | | | | | | | | | | |
| TEA Laurylsulfat | | | | | | | | | | | |
| Emulsifieres/Stabiliser | | | | | | | | | | | |
| Dermofeel Easymuls Plus | | | | | | | | | | | |
| Dermosoft Octiol | | | | | | | | | | | |
| Versatil MPC | | | | | | | | | | | |
| Antilsoft SC | | | | | | | | | | | |
| Oxypon 401 | | | | | | | | | | | |
| Glycerin pflanzlich 99,5% | | | | | | | | | | | |
| Polyglycerin-6 | | | | | | | | | | | |
| Cetiol OE | | | | | | | | | | | |
| Glucopon 650 EC/HH | | | | | | | | | | | |
| Cosmacol ELI | | | | | | | | | | | |
| Tegosoft PC 31 | 1.50 | 1.50 | 1.50 | 1.50 | 1.50 | 1.50 | 1.50 | 1.50 | 1.50 | 1.50 | 1.50 |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | 1.50 | 1.50 | 1.50 | 1.50 | 1.50 | 1.50 | 1.50 | 1.50 | 1.50 |
| pH Adjuster | 0.45 | 0.45 | 0.45 | 0.45 | 0.45 | 0.45 | 0.45 | 0.45 | 0.45 | 0.45 | 0.45 |
| Mononatriumphosphat calc. | | | | | | | | | | | |
| Sodiumchlorid solution 15% | | | | | | | | | | | |
| Performance | | | | | | | | | | | |
| Cleansing effect | 95% | 65% | 65% | 65% | 65% | 65% | 95% | 71% | 71% | 71% | 71% |
| Foamability | 3.0 | 4.0 | 4.0 | 4.0 | 4.0 | 4.0 | 3.0 | 4.5 | 4.5 | 4.5 | 4.5 |
| Stability | + | + | + | + | + | + | + | + | + | + | + |
| Trade Name | B11 | B12 | B13 | B14 | B15 | B16 | B17 | B18 | B19 | B20 | B21 |
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | 0.30 | 0.30 | 0.30 | 0.30 | 0.30 | 0.30 | 0.30 | 0.30 | 0.30 | 0.30 | 0.30 |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | | | | | | | | | | | |
| Actigum VSX 20 | | | | | | | | | | | |
| Kelcogel CG-LA | | | | | | | | | | | |
| Rhodicare S | | | | | | | | | | | |
| Walocel CRT 50.000 PA 07 | | | | | | | | | | | |
| Nonionics | | | | | | | | | | | |
| Gluco Tain Clear | 3.30 | 3.30 | 3.30 | 3.30 | 3.30 | | | | | | |
| Gluco Tain Care | | | | | | 2.88 | 1.92 | | | | |
| Rewopal LA 6 | | | | | | | 4.00 | 4.00 | 4.00 | 4.00 | 4.00 |
| Marlipal 24/70 | | | | | | | | | | | |
| Mackamine LA | | | | | | | | | | | |
| Bio Saponins | | | | | | | | | | | 4.00 |
| Steposol MET 10 U | | | | | | | | | | | |
| Betaine Coco Base | | | | | | | | | | | |
| Anionics | | | | | | | | | | | |
| SLES | 5.20 | 5.20 | 5.20 | 5.20 | 5.20 | 3.38 | 6.50 | 4.68 | 6.50 | 6.50 | 6.50 |
| Sulfated Castor Oil 75 WS | 3.00 | 3.00 | 3.00 | 3.00 | 4.00 | 3.00 | 3.00 | 3.00 | 3.00 | 3.00 | 3.00 |
| Rewopol SBF A 30 B | | | | | | | | 2.10 | 2.10 | | |
| Protelan LS9011 | | | | | | | | | | 1.80 | |
| Greenbentin MLS/100 | | | | | | | | | | | |
| Eucarol Age SS | | | | | | | | | | | |
| TEA Laurylsulfat | | | | | | | | | | | |
| Emulsifieres/Stabiliser | | | | | | | | | | | |
| Dermofeel Easymuls Plus | | | | | | | | | | | |
| Dermosoft Octiol | | | | | | | | | | | |
| Versatil MPC | | | | | | | | | | | |
| Antilsoft SC | | | | | | | | | | | |
| Oxypon 401 | | | | | | | | | | | |
| Glycerin pflanzlich 99,5% | | | | | | | | | | | |
| Polyglycerin-6 | | | | | | | | | | | |
| Cetiol OE | | | | | | | | | | | |
| Glucopon 650 EC/HH | | | | | | | | | | | |
| Cosmacol ELI | | | | | | | | | | | |
| Tegosoft PC 31 | 1.00 | 2.00 | 1.50 | 1.50 | 1.50 | 1.50 | 1.50 | 1.50 | 1.50 | 1.50 | 1.50 |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | 1.00 | 2.00 | 1.50 | 1.50 | 1.50 | 1.50 | 1.50 | 1.50 | 1.50 |
| pH Adjuster | 0.45 | 0.45 | 0.45 | 0.45 | 0.45 | 0.45 | 0.45 | 0.45 | 0.45 | 0.45 | 0.45 |
| Mononatriumphosphat calc. | | | | | | | | | | | |
| Sodiumchlorid solution 15% | | | | | | | | | | | |
| Performance | | | | | | | | | | | |
| Cleansing effect | 71% | 71% | 71% | 7I.% | 71% | 71% | 95% | 85% | 85% | 85% | 90% |
| Foamability | 4.5 | 4.5 | 4.5 | 4.5 | 4.5 | 4.5 | 3.0 | 3.0 | 3.0 | 3.0 | 3.0 |
| Stability | + | + | + | + | + | + | + | + | + | − | − |
| Trade Name | B22 | B23 | B24 | B25 | B26 | B27 | B28 | B29 | B30 | B31 | B32 |
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | 0.30 | 0.30 | 0.30 | 0.30 | 0.30 | 0.30 | 0.30 | 0.30 | 0.30 | 0.30 | 0.30 |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | | | | | | | | | | | |
| Actigum VSX 20 | | | | | | | | | | | |
| Kelcogel CG-LA | | | | | | | | | | | |
| Rhodicare S | | | | | | | | | | | |
| Walocel CRT 50.000 PA 07 | | | | | | | | | | | |
| Nonionics | | | | | | | | | | | |
| Gluco Tain Clear | | | | | | | | | | | |
| Gluco Tain Care | | | | | | | | | | | |
| Rewopal LA 6 | 4.00 | 4.00 | 6.00 | 6.00 | 6.00 | 6.00 | 6.00 | 6.00 | 6.00 | 6.00 | 6.00 |
| Marlipal 24/70 | | | | | | | | | | | |
| Mackamine LA | | | 6.00 | 4.00 | | | | | | | |
| Bio Saponins | | | | | | | | | | | |
| Steposol MET 10 U | | | | | | | | | | | |
| Betaine Coco Base | | | | | | | | | | | |
| Anionics | | | | | | | | | | | |
| SLES | 6.40 | 6.50 | 6.50 | 3.64 | 3.6 | 3.64 | 3.64 | 3.6 | 3.64 | 3.64 | 3.64 |
| Sulfated Castor Oil 75 WS | 3.00 | 3.00 | 3.00 | 3.00 | 3.00 | 3.00 | 3.00 | 3.00 | 3.00 | 3.00 | 3.00 |
| Rewopol SBF A 30 B | | | | 2.10 | 2.10 | 2.10 | 2.10 | 2.10 | 2.10 | 2.10 | 2.10 |
| Protelan LS9011 | | | | | | | | | | | |
| Greenbentin MLS/100 | | | | | | | | | | | |
| Eucarol Age SS | 1.80 | | | | | | | | | | |
| TEA Laurylsulfat | | | | | | | | | | | |
| Emulsifieres/Stabiliser | | | | | | | | | | | |
| Dermofeel Easymuls Plus | | | | | | | | | | | |
| Dermosoft Octiol | | | | | | | | | | | |
| Versatil MPC | | | | | | | | | | | |
| Antilsoft SC | | | | | | | | | | | |
| Oxypon 401 | | | | | | | | | | | |
| Glycerin pflanzlich 99,5% | | | | | | | | | | | |
| Polyglycerin-6 | | | | | | | | | | | |
| Cetiol OE | | | | | | | | 1.00 | 1.00 | 1.50 | 2.00 |
| Glucopon 650 EC/HH | | | | | | | | | | | |
| Cosmacol ELI | | | | | | | | | | | |
| Tegosoft PC 31 | 1.50 | 1.50 | 1.50 | 1.50 | 1.50 | 1.50 | 1.50 | 1.50 | 1.50 | 1.50 | 1.50 |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | 1.50 | 1.50 | 1.00 | 0.50 | | 1.50 | 1.50 | 1.50 | 1.50 |
| pH Adjuster | 0.45 | 0.45 | 0.45 | 0.45 | 0.45 | 0.45 | 0.45 | 0.45 | 0.45 | 0.45 | 0.45 |
| Mononatriumphosphat calc. | | | | | | | | | | | |
| Sodiumchlorid solution 15% | | | | | | | | | | | |
| Performance | | | | | | | | | | | |
| Cleansing effect | 95% | 95% | 95% | 95% | 95% | 95% | 95% | 95% | 95% | 95% | 95% |
| Foamability | 3.0 | 3.0 | 3.0 | 3.0 | 3.0 | 3.0 | 3.0 | 3.0 | 3.0 | 3.0 | 3.0 |
| Stability | − | − | − | + | − | − | − | + | + | + | + |
| Trade Name | B33 | B34 | B35 | B36 | B37 | B38 | B39 | B40 | B41 | B42 | B43 |
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | 0.30 | 0.30 | 0.30 | 0.30 | 0.30 | 0.30 | 0.30 | 0.30 | 0.30 | 0.30 | 0.30 |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | | | | | | | | | | | |
| Actigum VSX 20 | | | | | | | | | | | |
| Kelcogel CG-LA | | | | | | | | | | | |
| Rhodicare S | | | | | | | | | | | |
| Walocel CRT 50.000 PA 07 | | | | | | | | | | | |
| Nonionics | | | | | | | | | | | |
| Gluco Tain Clear | | | | | | | | | | | |
| Gluco Tain Care | | | | | | | | | | | |
| Rewopal LA 6 | 6.00 | 6.00 | 6.00 | 6.00 | 6.00 | 6.00 | 6.00 | 6.00 | 6.00 | 6.00 | 6.00 |
| Marlipal 24/70 | | | | | | | | | | | |
| Mackamine LA | | | | | | | | | | | |
| Bio Saponins | | | | | | | | | | | |
| Steposol MET 10 U | | | | | | | | | | | |
| Betaine Coco Base | | | | | | | | | | | |
| Anionics | | | | | | | | | | | |
| SLES | 3.6 | 3.64 | 3.6 | 3.64 | 3.64 | 3.64 | 3.64 | 3.64 | 3.64 | 3.64 | 3.64 |
| Sulfated Castor Oil 75 WS | 3.00 | 3.00 | 3.00 | 3.00 | 3.00 | 3.00 | 3.00 | 3.00 | 3.00 | 3.00 | 3.00 |
| Rewopol SBF A 30 B | 2.42 | 2.38 | 2.35 | 2.35 | 2.31 | 2.28 | 2.10 | 2.10 | 2.10 | 2.10 | 2.10 |
| Protelan LS9011 | | | | | | | | | | | |
| Greenbentin MLS/100 | | | | | | | | | | | |
| Eucarol Age SS | | | | | | | | | | | |
| TEA Laurylsulfat | | | | | | | | | | | |
| Emulsifieres/Stabiliser | | | | | | | | | | | |
| Dermofeel Easymuls Plus | | | | | | | 1.50 | | | 1.00 | 0.70 |
| Dermosoft Octiol | | | | | | | | | | | |
| Versatil MPC | | | | | | | | | | | |
| Antilsoft SC | | | | | | | | | | | |
| Oxypon 401 | | | | | | | | | | | |
| Glycerin pflanzlich 99,5% | | | | | | | | 1.50 | | | |
| Polyglycerin-6 | | | | | | | | | 1.50 | | |
| Cetiol OE | | | | | | | | | | | |
| Glucopon 650 EC/HH | | | | | | | | | | | |
| Cosmacol ELI | | | | | | | | | | | |
| Tegosoft PC 31 | 0.60 | 0.70 | 0.80 | 0.80 | 0.90 | 1.00 | | | | | |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | 1.50 | 1.50 | 1.50 | 1.50 | 1.50 | 1.50 | 1.50 | 1.50 | 1.50 |
| pH Adjuster | 0.45 | 0.45 | 0.45 | 0.45 | 0.45 | 0.45 | 0.45 | 0.45 | 0.45 | 0.45 | 0.45 |
| Mononatriumphosphat calc. | | | | | | | | | | | |
| Sodiumchlorid solution 15% | | | | | | | | | | | |
| Performance | | | | | | | | | | | |
| Cleansing effect | 95% | 95% | 95% | 95% | 95% | 95% | 95% | 95% | 95% | 95% | 95% |
| Foamability | 3.0 | 3.0 | 3.0 | 3.0 | 3.0 | 3.0 | 3.0 | 3.0 | 3.0 | 3.0 | 3.0 |
| Stability | − | − | − | − | − | − | + | − | − | + | + |
| Trade Name | B44 | B45 | B46 | B47 | B48 | B49 | B50 | B51 | B52 | B53 | B54 |
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | 0.30 | 0.30 | 0.30 | 0.30 | 0.30 | 0.30 | 0.30 | 0.30 | 0.30 | 0.30 | 0.30 |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | | | | | | | | | | | |
| Actigum VSX 20 | | | | | | | | | | | |
| Kelcogel CG-LA | | | | | | | | | | | |
| Rhodicare S | | | | | | | | | | | |
| Walocel CRT 50.000 PA 07 | | | | | | | | | | | |
| Nonionics | | | | | | | | | | | |
| Gluco Tain Clear | | | | | | | | | | | |
| Gluco Tain Care | | | | | | | | | | | |
| Rewopal LA 6 | 6.00 | 6.00 | 6.00 | 6.00 | 6.00 | 6.00 | 6.00 | 6.00 | 6.00 | 6.00 | 6.00 |
| Marlipal 24/70 | | | | | | | | | | | |
| Mackamine LA | | | | | | | | | | | |
| Bio Saponins | | | | | | | | | | | |
| Steposol MET 10 U | | | | | | | | | | | |
| Betaine Coco Base | | | | | | | | | | | |
| Anionics | | | | | | | | | | | |
| SLES | 3.64 | 3.64 | 3.64 | 3.64 | 3.64 | 3.64 | 3.64 | 3.64 | 3.64 | 3.64 | 3.64 |
| Sulfated Castor Oil 75 WS | 3.00 | 3.00 | 3.00 | 3.00 | 3.00 | 3.00 | 3.00 | 3.00 | 3.00 | 3.00 | 3.00 |
| Rewopol SBF A 30 B | 2.10 | 2.10 | 2.10 | 2.10 | 2.10 | 2.10 | 2.10 | 2.10 | 2.10 | 2.10 | 2.10 |
| Protelan LS9011 | | | | | | | | | | | |
| Greenbentin MLS/100 | | | | | | | | | | | |
| Eucarol Age SS | | | | | | | | | | | |
| TEA Laurylsulfat | | | | | | | | | | | |
| Emulsifieres/Stabiliser | | | | | | | | | | | |
| Dermofeel Easymuls Plus | 0.50 | 0.40 | 0.30 | 0.20 | | | | | | | |
| Dermosoft Octiol | | | | | 0.50 | 1.00 | 1.50 | | | | |
| Versatil MPC | | | | | | | | 0.50 | 1.00 | 1.50 | |
| Antilsoft SC | | | | | | | | | | | 1.00 |
| Oxypon 401 | | | | | | | | | | | |
| Glycerin pflanzlich 99,5% | | | | | | | | | | | |
| Polyglycerin-6 | | | | | | | | | | | |
| Cetiol OE | | | | | | | | | | | |
| Glucopon 650 EC/HH | | | | | | | | | | | |
| Cosmacol ELI | | | | | | | | | | | |
| Tegosoft PC 31 | | | | | 1.50 | 1.50 | 1.50 | 1.50 | 1.50 | 1.50 | |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | 1.50 | 1.50 | 1.50 | 1.50 | 1.50 | 1.50 | 1.50 | 1.50 | 1.50 |
| pH Adjuster | 0.45 | 0.45 | 0.45 | 0.45 | 0.45 | 0.45 | 0.45 | 0.45 | 0.45 | 0.45 | 0.45 |
| Mononatriumphosphat calc. | | | | | | | | | | | |
| Sodiumchlorid solution 15% | | | | | | | | | | | |
| Performance | | | | | | | | | | | |
| Cleansing effect | 95% | 95% | 95% | 95% | 95% | 95% | 95% | 95% | 95% | 95% | 95% |
| Foamability | 3.0 | 3.0 | 3.0 | 3.0 | 3.0 | 3.0 | 3.0 | 3.0 | 3.0 | 3.0 | 3.0 |
| Stability | − | − | − | − | + | + | + | + | + | + | + |
| Trade Name | B55 | B56 | B57 | B58 | B59 | B60 | B61 | B62 | B63 | Cl | C2 |
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | 0.30 | 0.30 | 0.30 | 0.30 | 0.30 | 0.30 | 0.30 | 0.30 | 0.30 | 0.40 | 0.40 |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | | | | | | | | | | | |
| Actigum VSX 20 | | | | | | | | | | | |
| Kelcogel CG-LA | | | | | | | | | | | |
| Rhodicare S | | | | | | | | | | | |
| Walocel CRT 50.000 PA 07 | | | | | | | | | | | |
| Nonionics | | | | | | | | | | | |
| Gluco Tain Clear | | | | | | | | | | 3.30 | 3.30 |
| Gluco Tain Care | | | | | | | | | | | |
| Rewopal LA 6 | 6.00 | 6.00 | 6.00 | 6.00 | 6.00 | | | | | | |
| Marlipal 24/70 | | | | | | 6.00 | 6.00 | 6.00 | 6.00 | | |
| Mackamine LA | | | | | | | | | | | |
| Bio Saponins | | | | | | | | | | | |
| Steposol MET 10 U | | | | | | | | | | | |
| Betaine Coco Base | | | | | | | | | | | |
| Anionics | | | | | | | | | | | |
| SLES | 3.64 | 3.64 | 3.64 | 3.64 | 3.64 | 3.64 | 3.64 | 3.64 | 3.64 | 3.38 | 5.20 |
| Sulfated Castor Oil 75 WS | 3.00 | 3.00 | 3.00 | 3.00 | 3.00 | 3.00 | 3.00 | 3.00 | 3.00 | 3.00 | 3.00 |
| Rewopol SBF A 30 B | 2.10 | 2.10 | 2.10 | 2.10 | 2.10 | 2.10 | 2.28 | 2.45 | 2.63 | 1.75 | |
| Protelan LS9011 | | | | | | | | | | | |
| Greenbentin MLS/100 | | | | | | | | | | | |
| Eucarol Age SS | | | | | | | | | | | |
| TEA Laurylsulfat | | | | | | | | | | | |
| Emulsifieres/Stabiliser | | | | | | | | | | | |
| Dermofeel Easymuls Plus | | | | | | | | | | | |
| Dermosoft Octiol | | | | | | | | | | | |
| Versatil MPC | | | | | | | | | | | |
| Antilsoft SC | 0.80 | 0.50 | | | | | | | | | |
| Oxypon 401 | | | 1.50 | 2.25 | 3.00 | | | | | | |
| Glycerin pflanzlich 99,5% | | | | | | | | | | | |
| Polyglycerin-6 | | | | | | | | | | | |
| Cetiol OE | | | | | | | | | | | |
| Glucopon 650 EC/HH | | | | | | | | | | | |
| Cosmacol ELI | | | | | | | | | | | |
| Tegosoft PC 31 | | | | | | 1.50 | 1.00 | 0.50 | | 1.00 | 1.50 |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | 1.50 | 1.50 | 1.50 | 1.50 | 1.50 | 1.50 | 1.50 | 1.50 | 1.50 |
| pH Adjuster | 0.45 | 0.45 | 0.45 | 0.45 | 0.45 | 0.45 | 0.45 | 0.45 | 0.45 | 0.45 | 0.45 |
| Mononatriumphosphat calc. | | | | | | | | | | | |
| Sodiumchlorid solution 15% | | | | | | | | | | | |
| Performance | | | | | | | | | | | |
| Cleansing effect | 95% | 95% | 95% | 95% | 95% | 85% | 95% | 95% | 95% | 71% | 72% |
| Foamability | 3.0 | 3.0 | 3.0 | 3.0 | 3.0 | 3.0 | 3.0 | 3.0 | 3.0 | 4.5 | 4.5 |
| Stability | − | − | − | − | − | − | + | + | − | + | + |
| Trade Name | C3 | C4 | C5 | C6 | C7 | C8 | D1 | D2 | D3 | D4 | D5 |
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | 0.40 | 0.40 | 0.40 | 0.40 | 0.40 | 0.40 | 0.50 | 0.50 | 0.50 | 0.50 | 0.50 |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | | | | | | | | | | | |
| Actigum VSX 20 | | | | | | | | | | | |
| Kelcogel CG-LA | | | | | | | | | | | |
| Rhodicare S | | | | | | | | | | | |
| Walocel CRT 50.000 PA 07 | | | | | | | | | | | |
| Nonionics | | | | | | | | | | | |
| Gluco Tain Clear | 3.30 | 3.30 | 3.30 | 3.30 | 3.30 | 3.30 | 2.20 | 2.20 | 2.20 | 2.75 | 2.75 |
| Gluco Tain Care | | | | | | | | | | | |
| Rewopal LA 6 | | | | | | | | | | | |
| Marlipal 24/70 | | | | | | | | | | | |
| Mackamine LA | | | | | | | | | | | |
| Bio Saponins | | | | | | | | | | | |
| Steposol MET 10 U | | | | | | | | | | | |
| Betaine Coco Base | | | | | | | | | | 1.86 | 1.86 |
| Anionics | | | | | | | | | | | |
| SLES | 3.38 | 3.38 | 3.38 | 3.38 | 3.38 | 3.38 | 3.90 | 3.90 | 3.38 | 5.20 | 3.38 |
| Sulfated Castor Oil 75 WS | 3.00 | 2.00 | 4.00 | 3.00 | 3.00 | 3.00 | 3.00 | 2.00 | 2.00 | 3.00 | 3.00 |
| Rewopol SBF A 30 B | 1.75 | 1.75 | 1.75 | 1.75 | 1.75 | 1.75 | 1.75 | 2.45 | 3.15 | | |
| Protelan LS9011 | | | | | | | | | | | |
| Greenbentin MLS/100 | | | | | | | | | | | |
| Eucarol Age SS | | | | | | | | | | | |
| TEA Laurylsulfat | | | | | | | | | | | |
| Emulsifieres/Stabiliser | | | | | | | | | | | |
| Dermofeel Easymuls Plus | | | | | | | | | | | |
| Dermosoft Octiol | | | | | | | | | | | |
| Versatil MPC | | | | | | | | | | | |
| Antilsoft SC | | | | | | | | | | | |
| Oxypon 401 | | | | | | | | | | | |
| Glycerin pflanzlich 99,5% | | | | | | | | | | | |
| Polyglycerin-6 | | | | | | | | | | | |
| Cetiol OE | | | | | | | | | | | |
| Glucopon 650 EC/HH | | | | | | | | | | | |
| Cosmacol ELI | | | | | | | | | | | |
| Tegosoft PC 31 | 1.50 | 1.50 | 1.50 | 1.50 | 1.50 | 2.00 | 1.50 | 1.50 | 1.00 | 1.50 | 1.50 |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | 1.50 | 1.00 | 2.00 | 1.50 | 1.50 | 1.50 | 1.50 | 1.50 | 1.50 |
| pH Adjuster | 0.45 | 0.45 | 0.45 | 0.45 | 0.45 | 0.45 | 0.45 | 0.45 | 0.45 | 0.50 | 0.45 |
| Mononatriumphosphat calc. | | | | | | | | | | | |
| Sodiumchlorid solution 15% | | | | | | | | | | | |
| Performance | | | | | | | | | | | |
| Cleansing effect | 71% | 72% | 73% | 73% | 73% | 73% | 71% | 72% | 74% | 75% | 75% |
| Foamability | 4.5 | 4.5 | 4.5 | 4.5 | 4.5 | 4.5 | 4.5 | 4.5 | 4.5 | 4.5 | 4.5 |
| Stability | + | + | + | + | + | + | + | + | + | + | + |
| Trade Name | D6 | D7 | D8 | D9 | D10 | D11 | D12 | D13 | D14 | D15 | D16 |
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | 0.50 | 0.50 | 0.50 | 0.50 | 0.50 | 0.50 | 0.50 | 0.50 | 0.50 | 0.50 | 0.50 |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | | | | | | | | | | | |
| Actigum VSX 20 | | | | | | | | | | | |
| Kelcogel CG-LA | | | | | | | | | | | |
| Rhodicare S | | | | | | | | | | | |
| Walocel CRT 50.000 PA 07 | | | | | | | | | | | |
| Nonionics | | | | | | | | | | | |
| Gluco Tain Clear | 2.75 | 3.30 | 3.30 | 3.30 | 3.30 | | | | | | |
| Gluco Tain Care | | | | | | | | | | | |
| Rewopal LA 6 | | | | | | 6.00 | 6.00 | 6.00 | 6.00 | 6.00 | 6.00 |
| Marlipal 24/70 | | | | | | | | | | | |
| Mackamine LA | | | | | | | | | | | |
| Bio Saponins | | | | | | | | | | | |
| Steposol MET 10 U | | | | | | | | | | | |
| Betaine Coco Base | 1.86 | | | | | | | | | | |
| Anionics | | | | | | | | | | | |
| SLES | 7.80 | 3.38 | 3.64 | 5.20 | 3.64 | 3.64 | 3.64 | 3.64 | 3.64 | 3.64 | 3.64 |
| Sulfated Castor Oil 75 WS | 3.00 | 3.00 | 3.00 | 3.00 | 3.00 | 3.00 | 3.00 | 3.00 | 3.00 | 3.00 | 3.00 |
| Rewopol SBF A 30 B | | 1.75 | 1.75 | | 1.75 | 2.10 | 2.10 | 2.35 | 2.31 | 2.28 | 2.45 |
| Protelan LS9011 | | | | | | | | | | | |
| Greenbentin MLS/100 | | | | | | | | | | | |
| Eucarol Age SS | | | | | | | | | | | |
| TEA Laurylsulfat | | | | | | | | | | | |
| Emulsifieres/Stabiliser | | | | | | | | | | | |
| Dermofeel Easymuls Plus | | | 1.50 | | | | | | | | |
| Dermosoft Octiol | | | | | | | | | | | |
| Versatil MPC | | | | | | | | | | | |
| Antilsoft SC | | | | | | | | | | | |
| Oxypon 401 | | | | | | | | | | | |
| Glycerin pflanzlich 99,5% | | | | | | | | | | | |
| Polyglycerin-6 | | | | | | | | | | | |
| Cetiol OE | | | | | | | | | | | |
| Glucopon 650 EC/HH | | | | | | | | | | | |
| Cosmacol ELI | | 1.50 | | | | | | | | | |
| Tegosoft PC 31 | 1.50 | | | 1.50 | 1.50 | 1.50 | 1.50 | 0.80 | 0.90 | 1.00 | 0.50 |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | 1.50 | 1.50 | 1.50 | 1.50 | 1.50 | 1.50 | 1.50 | 1.50 | 1.50 |
| pH Adjuster | 0.45 | 0.45 | 0.45 | 0.45 | 0.45 | 0.45 | 0.45 | 0.45 | 0.45 | 0.45 | 0.45 |
| Mononatriumphosphat calc. | | | | | | | | | | | |
| Sodiumchlorid solution 15% | | | | | | | | | | | |
| Performance | | | | | | | | | | | |
| Cleansing effect | 75% | 74% | 73% | 72% | 71% | 95% | 95% | 95% | 95% | 95% | 95% |
| Foamability | 4.5 | 4.5 | 5.0 | 5.0 | 4.5 | 3.0 | 3.0 | 3.0 | 3.0 | 3.0 | 3.0 |
| Stability | + | − | + | + | + | + | + | − | − | − | − |
| Trade Name | D17 | D18 | D19 | D20 | D21 | D22 | D23 | D24 | E1 | E2 | F1 |
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | 0.50 | 0.50 | 0.50 | 0.50 | 0.50 | 0.50 | 0.50 | 0.50 | 0.60 | 0.60 | 0.80 |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | | | | | | | | | | | |
| Actigum VSX 20 | | | | | | | | | | | |
| Kelcogel CG-LA | | | | | | | | | | | |
| Rhodicare S | | | | | | | | | | | |
| Walocel CRT 50.000 PA 07 | | | | | | | | | | | |
| Nonionics | | | | | | | | | | | |
| Gluco Tain Clear | | | | | | | | | 3.30 | 3.30 | 3.30 |
| Gluco Tain Care | | | | | | | | | | | |
| Rewopal LA 6 | 6.00 | 6.00 | 6.00 | | | | | | | | |
| Marlipal 24/70 | | | | | | | | | | | |
| Mackamine LA | | | | 10.00 | | | | | | | |
| Bio Saponins | | | | | | | | | | | |
| Steposol MET 10 U | | | | | | 4.00 | | | | | |
| Betaine Coco Base | | | 1.86 | 1.86 | 1.86 | 1.86 | 3.10 | | | | |
| Anionics | | | | | | | | | | | |
| SLES | 3.64 | 3.64 | 3.64 | 6.40 | 6.50 | 6.50 | 6.40 | 6.40 | 5.20 | 3.38 | 3.38 |
| Sulfated Castor Oil 75 WS | 3.00 | 3.00 | | 3.00 | 3.00 | 3.00 | 3.00 | 3.00 | 3.00 | 3.00 | 3.00 |
| Rewopol SBF A 30 B | 2.42 | 2.38 | | | | | | 3.50 | | 1.75 | |
| Protelan LS9011 | | | | | | | | | | | |
| Greenbentin MLS/100 | | | | | 4.00 | | | | | | |
| Eucarol Age SS | | | | | | | | | | | |
| TEA Laurylsulfat | | | 2.40 | | | | | | | | |
| Emulsifieres/Stabiliser | | | | | | | | | | | |
| Dermofeel Easymuls Plus | | | | | | | | | | | |
| Dermosoft Octiol | | | | | | | | | | | |
| Versatil MPC | | | | | | | | | | | |
| Antilsoft SC | | | | | | | | | | | |
| Oxypon 401 | | | | | | | | | | | |
| Glycerin pflanzlich 99,5% | | | | | | | | | | | |
| Polyglycerin-6 | | | | | | | | | | | |
| Cetiol OE | | | | | | | | | | | |
| Glucopon 650 EC/HH | | | | | | | | | | | |
| Cosmacol ELI | | | | | | | | | | | |
| Tegosoft PC 31 | 0.60 | 0.70 | 1.50 | 1.50 | 1.50 | 1.50 | 1.50 | 1.50 | 1.50 | 1.50 | 1.50 |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | | 1.50 | 1.50 | 1.50 | 1.50 | 1.50 | 1.50 | 1.50 | 1.50 |
| pH Adjuster | 0.45 | 0.45 | 0.45 | 0.45 | 0.45 | 0.45 | 0.45 | 0.45 | 0.45 | 0.45 | 0.45 |
| Mononatriumphosphat calc. | | | | | | | | | | | |
| Sodiumchlorid solution 15% | | | | | | | | | | | |
| Performance | | | | | | | | | | | |
| Cleansing effect | 95% | 95% | 95% | 65% | 65% | 65% | 65% | 65% | 72% | 74% | 75% |
| Foamability | 3.0 | 3.0 | 3.0 | 3.0 | 3.0 | 3.0 | 3.0 | 3.0 | 3.0 | 4.0 | 4.0 |
| Stability | − | − | − | − | − | − | − | − | + | + | + |
| Trade Name | F2 | F3 | G1 | H1 | I1 | J1 | K1 | L1 | L2 | L3 | L4 |
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | 0.80 | 0.80 | 0.04 | | | | | | | | |
| Sucraclear HC-31 | | | | 0.30 | | | | | | | |
| Jungbunzlauer FEDCS-PC | | | | | 0.30 | | | | | | |
| Simulgel FL | | | | | | 2.00 | | | | | |
| Exilva | | | | | | | 3.00 | | | | |
| Aristoflex TAC | | | | | | | | 0.50 | 0.60 | 0.70 | 1.00 |
| Actigum VSX 20 | | | | | | | | | | | |
| Kelcogel CG-LA | | | 0.08 | | | | | | | | |
| Rhodicare S | | | | | | | | | | | |
| Walocel CRT 50.000 PA 07 | | | | | | | | | | | |
| Nonionics | | | | | | | | | | | |
| Gluco Tain Clear | 3.30 | 3.30 | 3.30 | | | | | | | | |
| Gluco Tain Care | | | | | | | | | | | |
| Rewopal LA 6 | | | | 6.00 | 6.00 | 6.00 | 6.00 | 6.00 | 6.00 | 6.00 | 6.00 |
| Marlipal 24/70 | | | | | | | | | | | |
| Mackamine LA | | | | | | | | | | | |
| Bio Saponins | | | | | | | | | | | |
| Steposol MET 10 U | | | | | | | | | | | |
| Betaine Coco Base | | | | | | | | | | | |
| Anionics | | | | | | | | | | | |
| SLES | 3.38 | 3.38 | 3.38 | 3.64 | 3.64 | 3.64 | 3.64 | 3.64 | 3.64 | 3.64 | 3.64 |
| Sulfated Castor Oil 75 WS | 3.00 | 3.00 | 3.00 | 3.00 | 3.00 | 3.00 | 3.00 | 3.00 | 3.00 | 3.00 | 3.00 |
| Rewopol SBF A 30 B | 1.75 | | | 2.10 | 2.10 | 2.10 | 2.10 | 2.10 | 2.10 | 2.10 | 2.10 |
| Protelan LS9011 | | | | | | | | | | | |
| Greenbentin MLS/100 | | | | | | | | | | | |
| Eucarol Age SS | | | | | | | | | | | |
| TEA Laurylsulfat | | | | | | | | | | | |
| Emulsifieres/Stabiliser | | | | | | | | | | | |
| Dermofeel Easymuls Plus | | | | | | | | | | | |
| Dermosoft Octiol | | | | | | | | | | | |
| Versatil MPC | | | | | | | | | | | |
| Antilsoft SC | | | | | | | | | | | |
| Oxypon 401 | | | | | | | | | | | |
| Glycerin pflanzlich 99,5% | | | | | | | | | | | |
| Polyglycerin-6 | | | | | | | | | | | |
| Cetiol OE | | | | | | | | | | | |
| Glucopon 650 EC/HH | | 8.00 | | | | | | | | | |
| Cosmacol ELI | | | | | | | | | | | |
| Tegosoft PC 31 | 1.50 | 1.50 | 1.50 | 1.50 | 1.50 | 1.50 | 1.50 | 1.50 | 1.50 | 1.50 | 1.50 |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | 1.50 | 1.50 | 1.50 | 1.50 | 1.50 | 1.50 | 1.50 | 1.50 | 1.50 |
| pH Adjuster | 0.45 | 0.45 | 0.45 | 0.45 | 0.45 | 0.45 | 0.45 | 0.45 | 0.45 | 0.45 | 0.45 |
| Mononatriumphosphat calc. | | | 0.10 | | | | | | | | |
| Sodiumchlorid solution 15% | | | 0.70 | | | | | | | | |
| Performance | | | | | | | | | | | |
| Cleansing effect | 74% | 75% | 75% | 95% | 95% | 95% | 95% | 95% | 95% | 95% | 95% |
| Foamability | 4.0 | 4.0 | 3.0 | 3.0 | 3.0 | 3.0 | 3.0 | 3.0 | 3.0 | 3.0 | 3.0 |
| Stability | + | + | − | + | + | + | − | − | − | − | + |
| Trade Name | L5 | M1 | M2 | M3 | M4 | M5 | M6 | N1 | O1 | O2 | O3 |
| Water | ad | ad | ad | ad | ad | ad | ad | ad | ad | ad | ad |
| | 100 | 100 | 100 | 100 | 100 | 100 | 100 | 100 | 100 | 100 | 100 |
| Yield improvement | | | | | | | | | | | |
| Keltrol CG-SFT | | | | | | | | | | | |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | 1.00 | | | | | | | | | | |
| Actigum VSX 20 | | 0.10 | 0.20 | 0.30 | 0.40 | 0.50 | 0.60 | | | | |
| Kelcogel CG-LA | | | | | | | | 0.10 | | | |
| Rhodicare S | | | | | | | | | 0.50 | | 0.50 |
| Walocel CRT 50.000 PA 07 | | | | | | | | | | 0.50 | 0.50 |
| Nonionics | | | | | | | | | | | |
| Gluco Tain Clear | | | | | | | | | 3.3 | 3.3 | 3.3 |
| Gluco Tain Care | | | | | | | | | | | |
| Rewopal LA 6 | 6.00 | 6.00 | 6.00 | 6.00 | 6.00 | 6.00 | 6.00 | 6.00 | | | |
| Marlipal 24/70 | | | | | | | | | | | |
| Mackamine LA | | | | | | | | | | | |
| Bio Saponins | | | | | | | | | | | |
| Steposol MET 10 U | | | | | | | | | | | |
| Betaine Coco Base | | | | | | | | | | | |
| Anionics | | | | | | | | | | | |
| SLES | 3.64 | 3.64 | 3.64 | 3.64 | 3.64 | 3.64 | 3.64 | 3.64 | 3.38 | 3.38 | 3.38 |
| Sulfated Castor Oil 75 WS | 3.00 | 3.00 | 3.00 | 3.00 | 3.00 | 3.00 | 3.00 | | 2.80 | 2.80 | 2.80 |
| Rewopol SBF A 30 B | 2.10 | 2.10 | 2.10 | 2.10 | 2.10 | 2.10 | 2.10 | 2.10 | 1.65 | 1.65 | 1.65 |
| Protelan LS9011 | | | | | | | | | | | |
| Greenbentin MLS/100 | | | | | | | | | | | |
| Eucarol Age SS | | | | | | | | | | | |
| TEA Laurylsulfat | | | | | | | | 2.40 | | | |
| Emulsifieres/Stabiliser | | | | | | | | | | | |
| Dermofeel Easymuls Plus | | | | | | | | | 1.5 | 1.5 | 1.5 |
| Dermosoft Octiol | | | | | | | | | | | |
| Versatil MPC | | | | | | | | | | | |
| Antilsoft SC | | | | | | | | | | | |
| Oxypon 401 | | | | | | | | | | | |
| Glycerin pflanzlich 99,5% | | | | | | | | | | | |
| Polyglycerin-6 | | | | | | | | | | | |
| Cetiol OE | | | | | | | | | | | |
| Glucopon 650 EC/HH | | | | | | | | | | | |
| Cosmacol ELI | | | | | | | | | | | |
| Tegosoft PC 31 | | 1.50 | 1.50 | 1.50 | 1.50 | 1.50 | 1.50 | 1.50 | | | |
| Preservative | | | | | | | | | | | |
| Organic acid | 0.60 | 0.60 | 0.60 | 0.60 | 0.60 | 0.60 | 0.60 | 0.60 | 0.60 | 0.60 | 0.60 |
| Organic acid | 0.30 | 0.30 | 0.30 | 0.30 | 0.30 | 0.30 | 0.30 | 0.30 | 0.30 | 0.30 | 0.30 |
| Scrubber | | | | | | | | | | | |
| Cornmeal | 7.50 | 7.50 | 7.50 | 7.50 | 7.50 | 7.50 | 7.50 | 7.50 | 7.50 | 7.50 | 7.50 |
| Olive Stone Granules | 4.00 | 4.00 | 4.00 | 4.00 | 4.00 | 4.00 | 4.00 | 4.00 | 4.00 | 4.00 | 4.00 |
| Auxillaries | | | | | | | | | | | |
| Fragrance | 0.20 | 0.20 | 0.20 | 0.20 | 0.20 | 0.20 | 0.20 | 0.20 | 0.20 | 0.20 | 0.20 |
| Palmera A1813 | 1.50 | 1.50 | 1.50 | 1.50 | 1.50 | 1.50 | 1.50 | | 1.50 | 1.50 | 1.50 |
| pH Adjuster | 0.45 | 0.45 | 0.45 | 0.45 | 0.45 | 0.45 | 0.45 | 0.45 | 0.45 | 0.45 | 0.45 |
| Mononatriumphosphat calc. | | | | | | | | 0.10 | | | |
| Sodiumchlorid solution 15% | | | | | | | | 0.50 | | | |
| Performance | | | | | | | | | | | |
| Cleansing effect | 95% | 95% | 95% | 95% | 95% | 95% | 95% | 95% | 74% | 74% | 74% |
| Foamability | 3.0 | 3.0 | 3.0 | 3.0 | 3.0 | 3.0 | 3.0 | 3.0 | 4.0 | 1.0 | 1.0 |
| Stability | − | − | − | − | − | − | − | − | + | − | + |
| Trade Name | O1.1 | O1.2 | O1.3 | | | | | | | | |
| Water | ad | ad | ad | | | | | | | | |
| | 100 | 100 | 100 | | | | | | | | |
| Yield improvement | | | | | | | | | | | |
| Keltrol CG-SFT | | | | | | | | | | | |
| Sucraclear HC-31 | | | | | | | | | | | |
| Jungbunzlauer FEDCS-PC | | | | | | | | | | | |
| Simulgel FL | | | | | | | | | | | |
| Exilva | | | | | | | | | | | |
| Aristoflex TAC | | | | | | | | | | | |
| Actigum VSX 20 | | | | | | | | | | | |
| Kelcogel CG-LA | | | | | | | | | | | |
| Rhodicare S | 0.45 | 0.40 | 0.35 | | | | | | | | |
| Walocel CRT 50.000 PA 07 | | | | | | | | | | | |
| Nonionics | | | | | | | | | | | |
| Gluco Tain Clear | 3.3 | 3.3 | 3.3 | | | | | | | | |
| Gluco Tain Care | | | | | | | | | | | |
| Rewopal LA 6 | | | | | | | | | | | |
| Marlipal 24/70 | | | | | | | | | | | |
| Mackamine LA | | | | | | | | | | | |
| Bio Saponins | | | | | | | | | | | |
| Steposol MET 10 U | | | | | | | | | | | |
| Betaine Coco Base | | | | | | | | | | | |
| Anionics | | | | | | | | | | | |
| SLES | 3.38 | 3.38 | 3.38 | | | | | | | | |
| Sulfated Castor Oil 75 WS | 2.80 | 2.80 | 2.80 | | | | | | | | |
| Rewopol SBF A 30 B | 1.65 | 1.65 | 1.65 | | | | | | | | |
| Protelan LS9011 | | | | | | | | | | | |
| Greenbentin MLS/100 | | | | | | | | | | | |
| Eucarol Age SS | | | | | | | | | | | |
| TEA Laurylsulfat | | | | | | | | | | | |
| Emulsifieres/Stabiliser | | | | | | | | | | | |
| Dermofeel Easymuls Plus | 1.5 | 1.5 | 1.5 | | | | | | | | |
| Dermosoft Octiol | | | | | | | | | | | |
| Versatil MPC | | | | | | | | | | | |
| Antilsoft SC | | | | | | | | | | | |
| Oxypon 401 | | | | | | | | | | | |
| Glycerin pflanzlich 99,5% | | | | | | | | | | | |
| Polyglycerin-6 | | | | | | | | | | | |
| Cetiol OE | | | | | | | | | | | |
| Glucopon 650 EC/HH | | | | | | | | | | | |
| Cosmacol ELI | | | | | | | | | | | |
| Tegosoft PC 31 | | | | | | | | | | | |
| Preservative | | | | | | | | | | | |
| Organic acid | 0.60 | 0.60 | 0.60 | | | | | | | | |
| Organic acid | 0.30 | 0.30 | 0.30 | | | | | | | | |
| Scrubber | | | | | | | | | | | |
| Cornmeal | 7.50 | 7.50 | 7.50 | | | | | | | | |
| Olive Stone Granules | 4.00 | 4.00 | 4.00 | | | | | | | | |
| Auxillaries | | | | | | | | | | | |
| Fragrance | 0.20 | 0.20 | 0.20 | | | | | | | | |
| Palmera A1813 | 1.50 | 1.50 | 1.50 | | | | | | | | |
| pH Adjuster | 0.45 | 0.45 | 0.45 | | | | | | | | |
| Mononatriumphosphat calc. | | | | | | | | | | | |
| Sodiumchlorid solution 15% | | | | | | | | | | | |
| Performance | | | | | | | | | | | |
| Cleansing effect | 74% | 74% | 74% | | | | | | | | |
| Foamability | 4.5 | 4.8 | 5.0 | | | | | | | | |
| Stability | + | + | + | | | | | | | | |

The data shows that using smooth flow xanthan gums and castor oil it is possible to produce stable foams containing scrubbing agents. Using conventional xanthan gums, increased the viscosity to above optional levels unless concentration of the gum is controlled. Using surfactants, such as one or more fatty acid glucamides, such as capryloyl/caproyl methyl glucamide improves the product as does the addition of other surfactants such as SLS and DLS. Changing the xanthan gum for other polysaccharide gums, such as mixtures of other gums or gellan gums affects the properties of the final product away from optimum results.

Further work with conventional xanthan gums showed that 0.5% xanthan gum was able to be used to produce a foam. However, when stored for over 14 days the viscosity increased leading to reduced foam production. Viscosities of over 3500 cP decreased foam production.

1. A foamable skin and hand cleansing composition comprising the following components based on the total composition of the cleansing composition:
a) 0.5 to 10% by weight of sulphated castor oil;
b) 0.1 to 1% by weight of xanthan gum;
c) 0 to 5% by weight of at least one non-aqueous solvent, typically an organic solvent;
d) 1 to 30% by weight of at least on surfactant;
e) 0 to 3% by weight of at least one emulsifier;
f) 1 to 20% by weight of at least one scrubbing agent;
g) 0 to 2% by weight of at least one preservative;
h) 0 to 2% by weight of at least one pH modifier;
i) 0 to 10% by weight of auxiliaries and/or additive; and
j) water to make up 100% by weight.

2. A composition according to claim 1, wherein the xanthan gum is smooth flow xanthan gum.

3. A composition according to claim 1, having a viscosity of 200 to 5000 mPa·s.

4. A composition according to claim 1, having a yield value of at least 30 dynes/cm².

5. A composition according to claim 1 having a pH of below 5.5.

6. A composition according to claim 1 comprising 1.0 to 8% by weight, more typically 3 to 5% by weight of sulphated castor oil a).

7. A composition according to claim 1 comprising 0.1 to 0.9% by weight of xanthan gum b).

8. A composition according to claim 7, wherein the xanthan gum is conventional xanthan gum and the amount of xanthan gum is 0.1 to 0.5% by weight.

9. A composition according to claim 1 comprising less than 3% by weight of non-aqueous solvent c).

10. A composition according to claim 1 comprising 2 to 20%, typically 3 to 10% by weight of surfactant d).

11. A composition according to claim 1 the surfactant d) is selected from one or more fatty acid glucamides, sodium laureth sulfate and disodium laureth sulfosuccinate and mixtures thereof.

12. A composition according to claim 1 comprising 0.5 to 2.5% by weight, typically 1.0 to 2.0% by weight emulsifier e).

13. A composition according to claim 1 comprising 5 to 15% by weight of scrubbing agent.

14. A composition according to claim 1 comprising less than 0.5 wt % of an additional thickener.

15. A hand pump foamer apparatus comprising a composition according to claim 1.

16. A method of washing skin of a subject comprising expelling a composition according to claim 1 through a foaming apparatus to produce a foam and applying the foam to a portion of skin of the subject.
